(19) United States
(12) Patent Application Publication
(10) Pub. No.: US 2025/0255682 A1
(43) Pub. Date: Aug. 14, 2025

(54) DEVICE, SYSTEM, AND METHOD FOR TISSUE IDENTIFICATION DURING ROBOTIC SURGERY

(71) Applicant: THE REGENTS OF THE UNIVERSITY OF CALIFORNIA, Oakland, CA (US)

(72) Inventors: Rory Geoghegan, Los Angeles, CA (US); Robert N. Candler, La Canada Flintridge, CA (US); Erik Patrick Dutson, Los Angeles, CA (US); Songping Sun, Los Angeles, CA (US); Jake Pensa, Los Angeles, CA (US); Adam Abiri, Irvine, CA (US)

(21) Appl. No.: 18/856,984

(22) PCT Filed: Apr. 21, 2023
(86) PCT No.: PCT/US2023/066045

§ 371 Date: Oct. 15, 2024

Related U.S. Application Data
(60) Provisional application No. 63/333,330, filed on Apr. 21, 2022.

(51) Int. Cl. A61B 34/32; A61B 17/28; A61B 34/20; A61B 34/30; A61B 90/00; G16H 50/70

(52) U.S. Cl. CPC A61B 34/32; A61B 17/28; A61B 34/20; A61B 90/06; G16H 50/70; A61B 2034/2061; A61B 2034/305; A61B 2090/064

(57) ABSTRACT

A device for tissue mechanical property detection during robotic surgery, comprising a sensor frame having proximal and distal ends and a length therebetween, a force sensor disposed along the length of the sensor frame, and a displacement sensor configured to measure a position of the sensor frame. Related systems and methods are also disclosed.

### CROSS-REFERENCE TO RELATED APPLICATIONS

This application claims priority to U.S. provisional application No. 63/333,330 filed on Apr. 21, 2022, incorporated herein by reference in its entirety.

### BACKGROUND OF THE INVENTION

The advent of minimally invasive surgical techniques in the late 1980s has led to decreased incision size, shorter hospital stays, decreased pain, and quicker return to the work force, representing great steps forward for surgeons, patients, and healthcare systems. Robot-assisted technology was developed to further overcome technical difficulties encountered during laparoscopic procedures, such as the persistent limitations of the 2D visual modality, the loss of degrees of freedom in dexterity and the amplification of hand tremors via the rigid instruments.

Approximately 99,000 robotic procedures are performed every year and this number has been experiencing consistent growth over the past decade. While robotic-assisted procedures could be feasibly performed for cancer treatment with comparable outcomes to conventional laparoscopic procedures, loss of tactile feedback is one of the major disadvantages associated with the robotic technology which has been consistently shown to reduce surgeon performance and increase the learning curve, particularly in less experienced operators. The loss of ability to easily and properly characterize the location and extent of tumors and other relevant structures such as lymph nodes and blood vessels is a critical factor that continues to have a negative impact on application of robotic surgical systems in many surgical procedures including but not limited to GI oncological procedures.

The rise of robotic surgery can be attributed to its ability to enhance surgical skills beyond conventional human capability. Miniature mechanical tools augment the human hand by providing minimally-invasive access with increased range of motion and flexible gesture scaling. High-definition endoscopes with fluorescence imaging capabilities enhance vision beyond that of the naked eye. Cognitive capability has been extended by the advent of real-time software algorithms that incorporate pre-operative imaging data to optimize surgical resection. All of these technological advances enhance the toolset available to the modern surgeon; however, in stark contrast, the sense of touch has been lost entirely. Thus, there is a need in the art to restore the sense of touch and indeed extend it beyond the capabilities of human mechanoreceptors. Such a system would be of great benefit for identifying structures that are usually identified by palpation during conventional open surgery e.g. soft tissue tumors.

### SUMMARY OF THE INVENTION

Some embodiments of the invention disclosed herein are set forth below, and any combination of these embodiments (or portions thereof) may be made to define another embodiment.

In one aspect, a device for tissue mechanical property detection during robotic surgery comprises a sensor frame having proximal and distal ends and a length therebetween; a force sensor disposed along the length of the sensor frame; and a displacement sensor configured to measure a position of the sensor frame.

In one embodiment, the device further comprises a temperature sensor.

In one embodiment, the device further comprises a loading puck near the distal end of the sensor frame.

In one embodiment, the sensor frame is configured as surgical forceps.

In one embodiment, the force sensor comprises one or more fiber Bragg grating (FBG) sensors.

In one embodiment, the force sensor comprises one or more piezoelectric sensors.

In one embodiment, the force sensor comprises one or more capacitive sensors.

In one embodiment, the force sensor comprises a multiplexed sensor.

In one embodiment, the displacement sensor comprises an angle encoder, a camera, or a stereoscope.

In another aspect, a system for tissue mechanical property detection during robotic surgery comprises a sensor frame having proximal and distal ends and a length therebetween; a force sensor disposed along the length of the sensor frame; a displacement sensor configured to measure a position of the sensor frame; and a robotic grasping arm, wherein the sensor frame is positioned as an end-effector of the robotic grasping arm.

In one embodiment, the system further comprises a computing system communicatively connected to the force and displacement sensors, comprising a processor and a non-transitory computer-readable medium with instructions stored thereon, which when executed by the processor, perform steps comprising: obtaining force data via the force sensor; obtaining tissue displacement data via the displacement sensor; applying the obtained force and displacement data to a tissue specific model representing the strain experienced by tissue in response to an external force; and identifying at least one mechanical property of the tissue based on the model output.

In another aspect, a method of identifying tissue mechanical properties during robotic surgery, comprising the steps of: providing the tissue mechanical property detection system as described above; grasping a tissue with the end-effector of the robotic grasping arm such that the sensor device engages the tissue; obtaining force data via the force sensor; obtaining tissue displacement data via the displacement sensor; applying the obtained force and displacement data to a tissue specific model representing the strain experienced by the tissue in response to an external force; and identifying at least one mechanical property of the tissue based on the model output.

In one embodiment, the method further comprises obtaining temperature data from a temperature sensor.

In one embodiment, the method further comprises identifying the type of tissue grasped based on the identification of the at least one tissue mechanical property.

In one embodiment, the at least one tissue mechanical property is determined based on a force-strain model.

In one embodiment, the identification of the type of tissue is based on comparing the identified tissue mechanical property against a library including tissue types and their mechanical properties, where the library is built via training data sets comprising a plurality of tissue types with known tissue properties.

In one embodiment, the method further comprises providing feedback to an end-user or autonomous system.

In one embodiment, the feedback comprises a vibration, a visual cue, or an auditory cue.

In one embodiment, the feedback is provided in less than 1 second, less than 0.5 seconds, or in less than 0.1 seconds after grasping the tissue.

In one embodiment, tissue displacement is inferred by recording the position of the graspers with a displacement sensor.

In one embodiments, the displacement sensor comprises an angle encoder, a camera, or a stereoscope.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1A depicts an integrated opto-mechanical force sensor in accordance with some embodiments.

FIG. 1B depicts a diagram showing an exemplary sensor design for tissue mechanical property detection during robotic surgery in accordance with some embodiments.

FIGS. 2A-2B depict a method for identifying tissue mechanical properties and/or classifying tissue during robotic surgery in accordance with some embodiments. FIG. 2A depicts utilizing sensorized forceps to manipulate tissue and a related feedback process. FIG. 2B depicts a flow-chart describing an exemplary method.

FIG. 3 depicts an experimental prototype of an exemplary sensor design for tissue mechanical property detection during robotic surgery integrated into a Da Vinci Bipolar Forceps instrument in accordance with some embodiments.

FIGS. 4A-4C depict a force calibration setup with Mark-10 force gauge and Fiber-Bragg-grating (FBG) integrated instrument with 3D printed clamping device in accordance with some embodiments.

FIG. 5A depicts the integrated sensor instrument of FIGS. 4A-4C grasping a silicone block in accordance with some embodiments.

FIG. 5B depicts a servo motor mounted on the base of the instrument of FIG. 1A to control the opening of the graspers in accordance with some embodiments.

FIG. 6 depicts the force calibration data for the strain sensor (FBG_2) and temperature sensor (FBG_T) of the instrument of FIG. 3 grasping the silicone block of FIG. 5A in accordance with some embodiments. The shift in Bragg wavelength of the strain sensor increased linearly with applied force while the temperature sensor is virtually unaffected by the applied force when compared to the strain sensor.

FIG. 7A depicts an exemplary force-strain curve derived from force and angle data of an FBG-integrated instrument in accordance with some embodiments.

FIG. 7B depicts experimental results of a force at 0.20 strain applied to silicon blocks of varying material in accordance with some embodiments. The sensor was able to differentiate between the four types of silicone that corresponds to their hardness consistently.

FIG. 8 depicts experimental results for the sorting of silicon blocks of varying material in accordance with some embodiments. Average error per subject per trial for each experiment group is shown where error bars show a 99% Confidence Interval. The performance using a Da Vinci surgical robot (DV) (Intuitive Surgical Inc., CA) was inferior to manual palpation (MP) while the FBG-integrated instrument successfully sorted all blocks without error.

FIGS. 9A-9D depict experimental results from silicone phantom tissue categorization experiments. In FIGS. 9A-9C solid lines represent library tissue models created from the training set. Blue markers are force-strain data from test samples. The three samples are Mold Star 20T (FIG. 9A), EcoFlex 00-10 (FIG. 9B), and Dragon Skin FX Pro (FIG. 9C). FIG. 9D shows average error per subject for manual palpation (MP) and the FBG-integrated instrument, error bars show 99% confidence Interval. Manual Palpation results in an average error rate of 38.4% while the FBG-integrated instrument successfully categorized all blocks without error.

FIG. 10 depicts an exemplary computing environment in which aspects of the invention may be practiced in accordance with some embodiments.

### DETAILED DESCRIPTION

It is to be understood that the figures and descriptions of the present invention have been simplified to illustrate elements that are relevant for a clear understanding of the present invention, while eliminating, for the purpose of clarity, many other elements found in related systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

Minimally invasive surgery (MIS) has been steadily gaining popularity in recent years due to smaller incisions which result in less blood loss, shorter hospital stays and faster recovery than conventional open surgery (Buess et al., Arch. Surg., vol. 135, no. 2, pp. 229-235, 2000; Brown et al., Stud. Health Technol. Inform., vol. 98, pp. 34-36, 2004; Lau et al., World J. Surg., vol. 21, no. 4, pp. 444-453, 1997). Robotic MIS (RMIS) was subsequently developed to further overcome technical difficulties encountered during laparoscopic procedures, such as the persistent limitations of the 2D visual modality, the loss of degrees of freedom in dexterity and the amplification of hand tremors via the rigid instruments (Schurr et al., Surg. Endosc., vol. 14, no. 4, pp. 375-381, April 2000; Moorthy et al., Surg. Endosc., vol. 18, no. 5, pp. 790-795, 2004; Munz et al., Surg. Endosc., vol. 18, no. 4, pp. 611-616, April 2004). RMIS is quickly gaining ground; a study in Michigan showed that robotic surgery constitutes 15.1% of all general surgery in 2018 (Sheetz et al., JAMA Netw. Open, vol. 3, no. 1, pp. e1918911-e1918911, January 2020). However, despite its popularity, robotic surgery also faces its challenges. While high equipment cost and additional training are some of the roadblocks to adopting robotic surgery, the loss of tactile feedback substantially impairs the surgeon's ability to easily and properly characterize the location and extent of tumors and other relevant structures such as lymph nodes and blood vessels that are not visually apparent (Li et al., Proc.-IEEE Int. Conf. Robot. Autom., pp. 5359-5364, 2012). For example, in an operation involving the Whipple procedure, tissue palpation can provide significant value during dissection of the portal vein from the posterior pancreatic surface, but palpation is not possible with current robotic technology (Hanly & Talamini, Am. J. Surg., vol. 188, no. 4A Suppl, pp. 19-26, 2004).

In the past decade, several groups have made progress in developing palpation tools in RMIS. However, current approaches (Mckinley et al., IEEE Int. Conf. Autom. Sci. Eng., vol. 2015-October, pp. 1151-1158 October 2015.; Perri et al., Int. J. Med. Robot., vol. 6, no. 2, pp. 211-220, June 2010; Xie et al., IEEE Int. Conf. Intell. Robot. Syst., pp. 2539-2544, 2013; Pacchierotti et al., IEEE Trans. Biomed. Eng., vol. 63, no. 2, pp. 278-287, February 2016) have focused on the development of dedicated palpation tools. For example, Mckinley et al (Mckinley et al., IEEE Int. Conf. Autom. Sci. Eng., vol. 2015-October, pp. 1151-1158 October 2015) developed a palpation probe based on a Hall Effect sensor which can detect hard subsurface structures via tissue indentation but only if the probe is aligned perpendicular to the tissue surface. The use of a dedicated tool greatly alters standard surgical practice which may degrade surgical performance as well as increase operating time and consequently cost. Moreover, the sensor itself contains numerous moving components which increase manufacturing complexity and thus cost. Finally, as with all other robotic palpation tools, the palpation probe only quantifies the applied force rather than the tissue mechanical properties. In order to infer a change in stiffness, the operator must attempt to achieve consistent indentation depth between measurements while ensuring the probe remains perpendicular to the tissue surface. This may be possible during a benchtop test on a flat tissue phantom but it is unrealistic during surgery. In addition to low cost and easy integration with existing surgical tools, a palpation tool would need to be sterilizable, water-proof and fit through trocars in order to be adopted in a clinical setting.

During open surgery, surgeons physically touch tissue with their hands and thus can gain insight based on the tissue mechanical properties. This enables them to identify tissue type, detect hard tumors buried in soft tissue, and detect subsurface structures such as blood vessels and nerves. However, during robotic and laparoscopic surgeries, surgeons interact with patient tissue only through surgical instruments, and therefore lose tactile information. This invention proposes an idea to acquire this information by detecting the mechanical properties of the tissue being handled by the instrument. The disclosed sensor device and system is able to be integrated into the surgical instrument for minimal footprint and ease of clinical adoption.

In some embodiments, the sensor utilizes force and/or strain sensors, such as Fiber-bragg-gratings (FBG), positioned in an optical fiber to detect the forces applied on the forceps or other end-effector during tissue grasping (the tissue contact force). The fiber is mounted on the end-effectors of a surgical instrument (forceps or similar) and enveloped in a flexible, biocompatible membrane material (Polydimethylsiloxane (PDMS) or similar).

In some embodiments, the device can identify tumors and other clinically significant structures (vessels, lymph nodes etc.) in real time during robotic or laparoscopic surgeries due to their different mechanical properties compared to surrounding tissues. The goal is to identify tissue with an accuracy at or beyond that which can be achieved using the human hand. This is achieved by using very sensitive force and displacement sensors and analyzing the data with software in real-time.

Disclosed herein is a sensor device that is able to be fully integrated into robotic surgical instrument systems and is capable of simultaneously quantifying the applied force and the tissue mechanical properties. In some embodiments, the sensor device is integrated into a surgical robot and configured to perform fully autonomous tissue characterization by analysis of sensor data and computer vision.

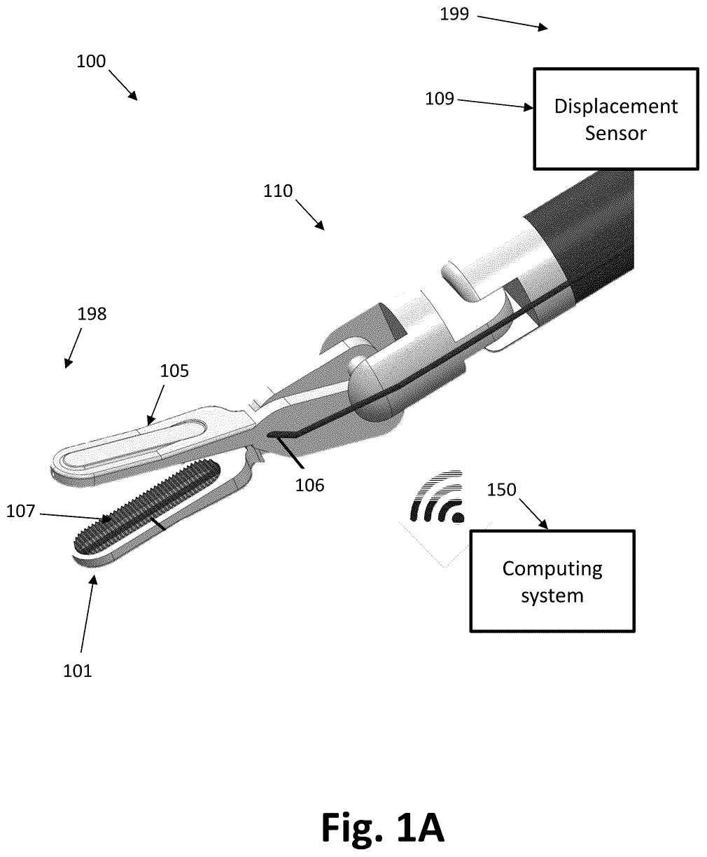

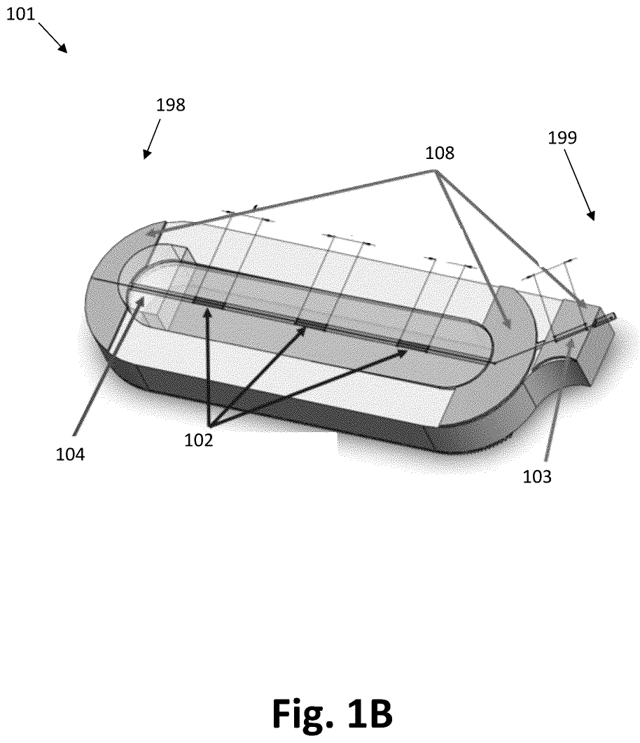

Referring now to FIGS. 1A-1B, an example integrated opto-mechanical force sensor device 101 and system 100 are shown. In some embodiments, the design includes an optical fiber 106 with one or more inscribed FBG sensors 102, wherein the optical fiber 106 is embedded in a flexible substrate or membrane 107 comprising a biocompatible material, such as PDMS or other silicone based materials for example, and directly placed in the end-effector, such as a forceps 105 for example, of a surgical robot arm. In some embodiments, additional sensors such as capacitive sensors, piezoresistive sensors, and/or piezoelectric sensors can be included to perform force measurements. In some embodiments, the force is quantified by embedding alternative sensors in the membrane such as capacitive or piezoelectric force sensors. In some embodiments, laser-photodiode pairs or similar can be included to perform transmittance, reflectance, absorbance or other suitable biospectroscopy measurements. In some embodiments, a shift in the reflective wavelengths of the sensor 102 is proportional to the axial strain on the fiber induced by the force applied to a tissue via the end-effector. By using this sensor design, tissue mechanical properties can be inferred from the force-strain curve estimated from the force applied to the tissue via the end-effector and angular or positional data of the end-effector as a proxy of tissue displacement. In some embodiments, the system 100 is configured to supply a force to a tissue in the range of about 0.01 N to 15 N. In some embodiments, the system 100 is configured to limit the force applied to the tissue to less than 2 N, or less than 1 N to mitigate accidental tissue crushing. For example and as detailed below, when using silicone blocks as tissue phantoms, the device 101 was able to successfully sort the blocks according to their hardness, and performed better than human subjects using non-sensorized surgical tools and manual palpation. In some embodiments, the device 101 is designed to be sterilizable, water-proof and have sufficiently low profile to fit through existing surgery trocars. In some embodiments, the device 101 which houses the sensors 102 has a length of about 5 mm to 30 mm, a width of about 2 mm to 15 mm, and a thickness of about 0.5 mm to 5 mm. Moreover, in some embodiments, the device 101 has the potential to surpass the tactile sensory capability of humans. In some embodiments, the measured tissue mechanical properties and subsequent tissue characterization can be used for decision making during RMIS or for autonomous surgery.

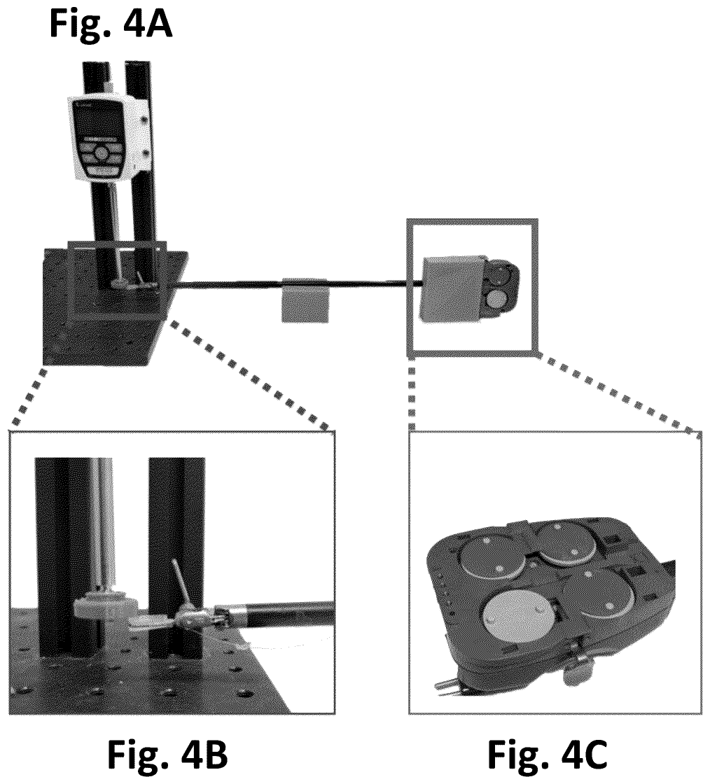

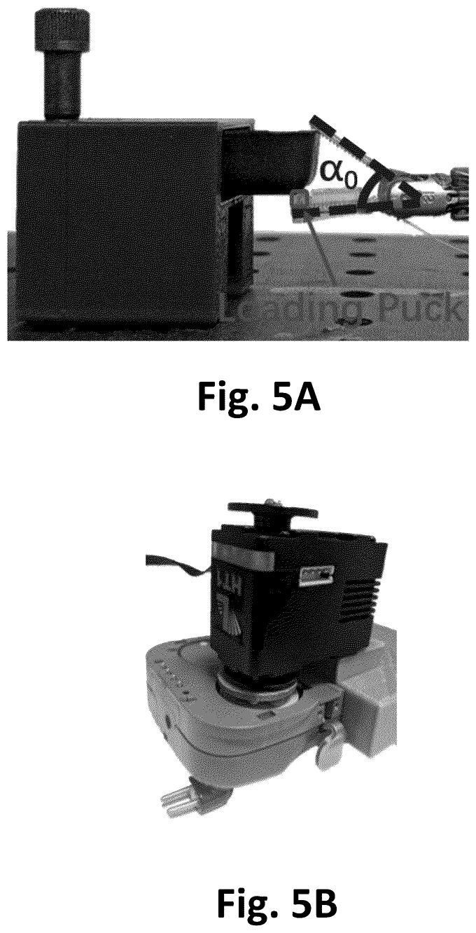

In some embodiments, the system 100 is configured for tissue mechanical property detection during robotic surgery and comprises a sensor frame 108 having proximal 199 and distal 198 ends and a length therebetween, a force sensor 102 disposed along the length of the sensor frame 108, a displacement sensor 109 configured to measure a position and/or angle of the sensor frame 108, and a robotic grasping arm 110, wherein the sensor frame 108 is positioned as an end-effector of the robotic grasping arm 110. In some embodiments, displacement is measured from the motors which drive the robot (FIG. 4C, FIG. 5B). In some embodiments, the ability to record the angle of the motor is standard for the surgical robot arm 110.

In some embodiments, the system 100 further includes a computing system 150 communicatively connected to the force 102 and displacement 109 sensors, comprising a processor and a non-transitory computer-readable medium with instructions stored thereon, which when executed by the processor, perform steps comprising obtaining force data via the force sensor 102, obtaining tissue displacement data via the displacement sensor 109, applying the obtained force and displacement data to a tissue specific model representing the strain experienced by tissue in response to an external force, and identifying at least one mechanical property of the tissue based on the model output.

### Smart Haptic Feedback

Smart haptic feedback aims to quantify tissue mechanical properties (e.g. Young's modulus) in real-time and alert the operator when the desired tissue is identified. For example, when searching for tumors in the bowel, the operator grasps suspicious tissue, the mechanical properties are quantified and if they are indicative of cancerous tissue a vibratory motor is activated beneath the operator's fingertips, a visual cue is shown and/or an auditory cue is produced. It is well known that such tumors are stiffer (increased Young's modulus) than healthy tissue; therefore, the smart haptic feedback system 100 can localize tumors based on a change in Young's modulus. This system 100 provides for improved tissue localization and consequently an increase in the accuracy of tumor resection. In order to quantify the tissue mechanical properties, both the force applied to the tissue and the resulting tissue displacement are recorded.

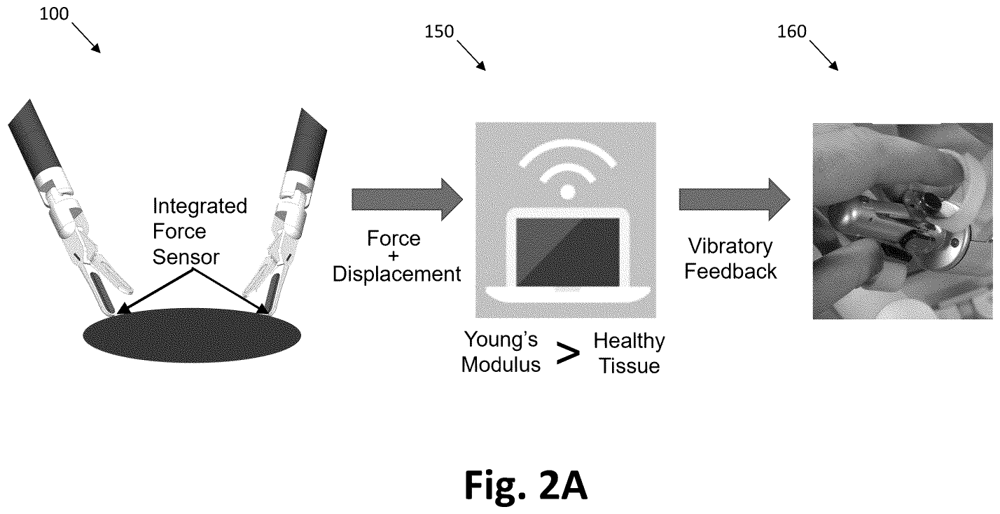

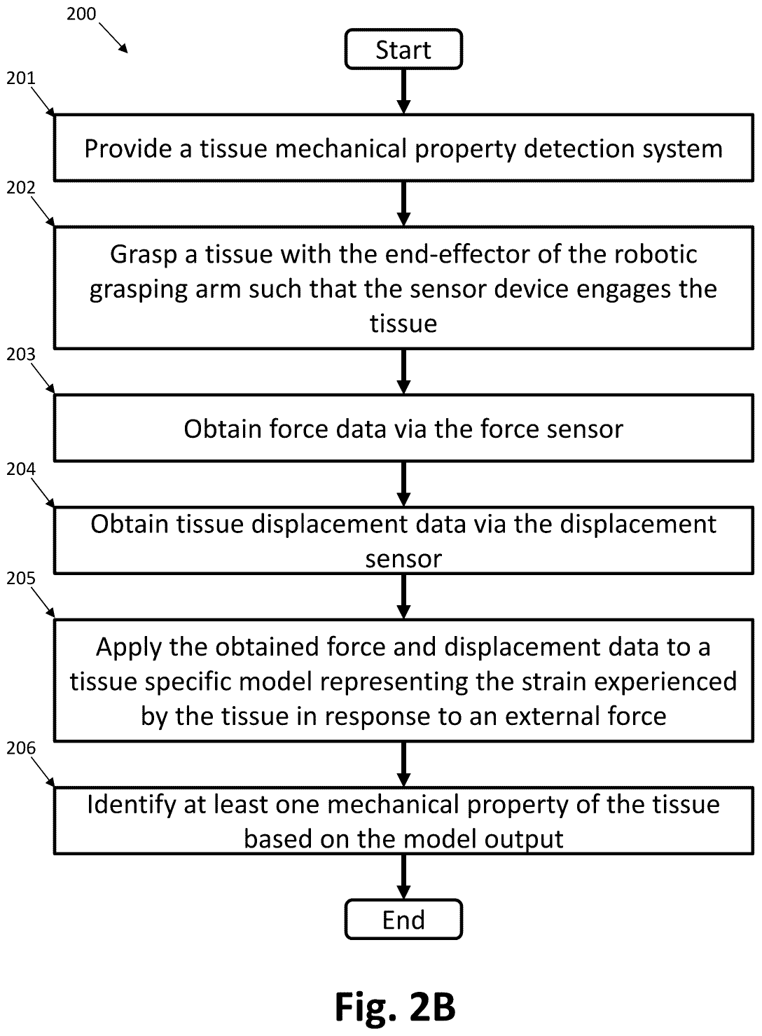

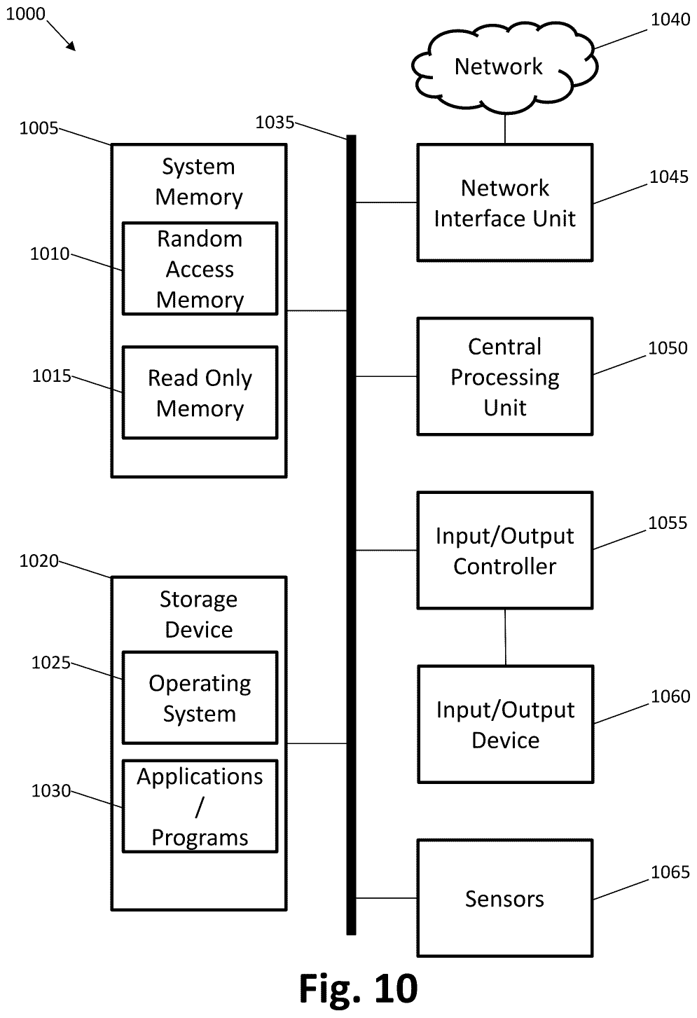

Referring now to FIGS. 2A, an exemplary process for utilizing system 100 to manipulate tissue and provide feedback 160 is shown. Force and displacement data from system 100 can be analyzed on a computing system 150 communicatively connected to the device 101 in real-time to quantify tissue mechanical properties (e.g. Young's modulus). An exemplary computing system is shown in FIG. 10 and described below. When a desired tissue is identified, a vibratory motor, visual cue, auditory cue, and/or similar haptic feedback device is activated on the surgical console to notify the user. Note, in this non-limiting example the desired tissue is a colorectal tumor which is known to exhibit a larger Young's modulus than healthy tissue.

### Integrated Force Sensor

In some embodiments, the force applied by the surgical instruments to the tissue is recorded via an integrated force and/or strain sensor 102 (FIGS. 1A-1B). In some embodiments, the sensor 102 comprises an optical fiber 106 with one or more integrated force or strain sensors 102 embedded in a biocompatible flexible membrane 107. The one or more integrated force and/or strain sensors 102 may comprise any suitable sensors including, but not limited to, FBGs, capacitive sensors and piezoresistive sensors. In some embodiments, during tissue grasping, the contact force is quantified by measuring the change in Bragg wavelength with an optical interrogator. The membrane may be manufactured from polydimethylsiloxane (PDMS) or other suitable flexible material.

### Tissue Displacement

In one example embodiment, during routine robotic tissue manipulation, forceps 105 are used as the end-effector to pinch the tissue by closing the graspers. The angle of the graspers is continuously recorded by the surgical platform via rotary encoders. Tissue displacement is quantified by measuring the change in angle after initial tissue contact as indicated by sensor 102. In a further embodiment, the tissue displacement is quantified by a dedicated rotary encoder or similar mounted on the instrument end-effectors. Alternatively, in another embodiment, tissue displacement may be quantified by real-time analysis of image data gathered by the robotic endoscope.

### Feedback to Operator

In some embodiments, the data from the integrated force sensor 102 and rotary encoders are analyzed in real-time to facilitate in vivo quantification of tissue mechanical properties. Tissue structures are then identified by comparing the results with known values for tissues of interest (e.g. colorectal tumors). When the desired tissue is identified, a software control module of the computing system 150 activates a feedback modality 160 on the surgical console. The feedback modality 160 may be an overlay on the surgical screen, an auditory cue, a vibratory motor, or any other suitable feedback modality. In some embodiments, the delay between tissue grasping and relaying feedback to the user is less than 1 second, less than 0.5 seconds, or less than 0.1 seconds. In some embodiments, the data can also be used as a real-time input for an autonomous surgery system. The feedback process is illustrated in FIG. 2A.

### Sensor Design

In some embodiments, the sensor device 101 is designed to collect force and displacement data during routine grasping motion in order for it to be used to detect tissue mechanical properties in a surgical setting. Specifically, in some example embodiments, force data is measured using FBG while displacement is measured through grasper angle.

In some embodiments, the device 101 is configured for tissue mechanical property detection during robotic surgery, and comprises a sensor frame 108 having proximal 199 and distal 198 ends and a length therebetween, a force sensor 102 disposed along the length of the sensor frame 108, and a displacement sensor 109 configured to measure a position of the sensor frame 108. In some embodiments, the device 101 further comprises a temperature sensor 103. In some embodiments, the temperature sensor 103 is positioned near the proximal end 199 of the sensor frame 108. In some embodiments, the device 101 further comprises a loading puck 104 near the distal end 198 of the sensor frame 108.

In some embodiments, the sensor frame 108 is configured as surgical forceps. In some embodiments the force sensor 102 comprises one or more fiber Bragg grating (FBG) sensors, one or more piezoelectric sensors, and/or one or more capacitive sensors. In some embodiments, the force sensor 102 comprises a multiplexed sensor. In some embodiments, the sensors are placed on and/or within any suitable portion and/or multiple portions of the end-effector including, but not limited to, a top portion, a bottom portion, a tip, a hinge, or a frame. In some embodiments, the displacement sensor 109 comprises an angle encoder, a camera, and/or a stereoscope. In some embodiments, the displacement sensor 109 can be positioned within device 101, proximate to device 101, be integral to and/or on the robotic grasping arm 110, and/or be integral to a control system for the robotic arm 110.

### Methods

Referring now to FIG. 2B, in some embodiments, a method 200 of identifying tissue mechanical properties during robotic surgery is shown. The method 200 starts at Operation 201 where the tissue mechanical property detection system 100 including device 101 as described above is provided. At Operation 202 a tissue is grasped with the end-effector of the robotic grasping arm such that the sensor device 101 engages the tissue. At Operation 203 force data is obtained via the force sensor 102. At Operation 204 tissue displacement data is obtained via the displacement sensor 109. In some embodiments, the force data and displacement data are obtained simultaneously. At Operation 205 the obtained force and displacement data is applied to a tissue specific model representing the strain experienced by the tissue in response to an external force. The method 200 ends at Operation 206 where at least one mechanical property of the tissue is identified based on the model output.

In some embodiments, the method 200 further includes obtaining temperature data from a temperature sensor 103. In some embodiments, the method 200 further includes identifying the type of tissue grasped based on the identification of the at least one tissue mechanical property. In some embodiments, the identification of the type of tissue is based on comparing the identified tissue mechanical property against a library including tissue types and their mechanical properties. In some embodiments, the library is built via training data sets of a plurality of tissue types with known tissue properties. In some embodiments, the at least one tissue mechanical property is determined based on a force-strain model.

In some embodiments, the method 200 further comprises providing feedback 160 to an end-user or autonomous system. In some embodiments, the feedback comprises a vibration, a visual cue, or an auditory cue. In some embodiments, the feedback is provided in less than 1 second, less than 0.5 seconds, or less than 0.1 seconds after grasping the tissue.

In some embodiments, tissue displacement is inferred by recording the position of the graspers with a displacement sensor 109. In some embodiments, the displacement sensor 109 comprises an angle encoder, a camera, or a stereoscope.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out exemplary embodiments of the present invention, and are not to be construed as limiting in any way the remainder of the disclosure.

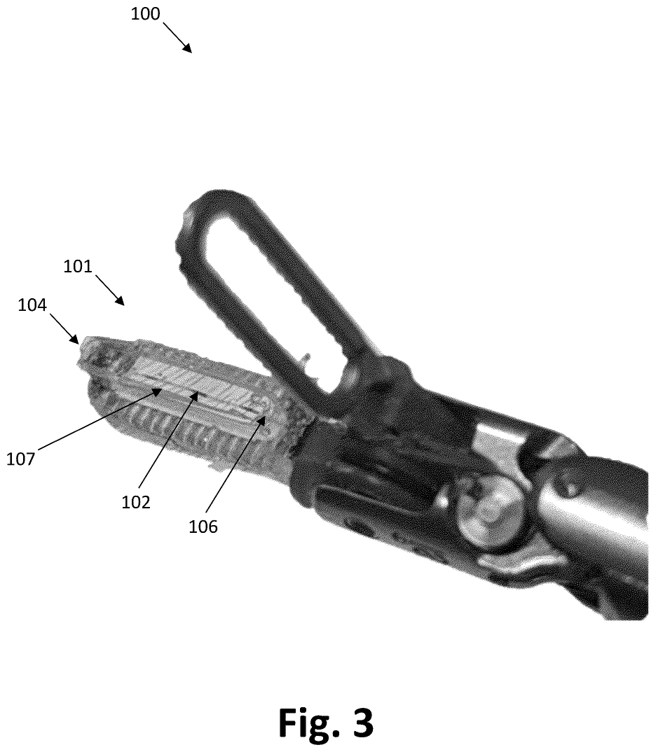

Referring now to FIGS. 3-8, experimental systems and results are shown. The sensing array 102 used in this study was custom manufactured by Technica & Femto Sensing International, LLC (GA, USA). It is constructed using a SMF-28-C optical fiber 106 (Corning Inc., NY, USA) coated with Polyimide with a 150 μm diameter and a 1 mm bend radius. Four FBG sensors 102 were inscribed in the distal section of the optical fiber 106, each with a length of 1 mm. Central wavelength for each FBG 102 was 1535, 1540, 1545, and 1550 nm. The strain induced change in wavelength was recorded using an off-the-shelf optical interrogator (FAZT-I4W (Technica & Femto Sensing International, LLC, GA, USA)), which had an absolute wavelength accuracy <3 μm, a wavelength precision <0.5 μm, and the scan frequency was 1 kHz.

The prototype sensor represents one but not all configurations (different center wavelengths, number of sensors, optical fiber type) of the design. The force sensors used in this design are also not limited to FBGs, but can include other types of force sensors such as piezoelectric sensor or capacitive sensors.

FIG. 3 shows the FBG fiber sensor device 101 integrated into a Da Vinci Si Fenestrated Bipolar Forceps (Intuitive Surgical Inc., California). The optical fiber 106 was fixed at both the distal and proximal end on one side of the end effector using steel-reinforced epoxy (J-B Weld 8276 KwikWeld Quick Setting Steel Reinforced Epoxy, J-B Weld, TX, USA) such that the first 3 FBGs 102 are located inside the end-effector to detect strain. The fourth FBG is encapsulated in steel-reinforced epoxy slightly above the joint of the end effector for maximum strain shielding needed for temperature measurement. The temperature sensor was needed to compensate the Bragg wavelength shift due to temperature fluctuations over room temperature to physiological temperature, to ensure rigorous performance in a surgical setting. The optical fiber 106 was integrated into the instrument using a degassed PDMS membrane 107 (Sylgard 184, 10:1 ratio (Dow Chemical Co. MI, USA)) which was cast with a 3D-printed mold and cured at 130° C. for 1 hour. The mold was designed so that the fiber within the end-effector is completely encapsulated in PDMS and a puck-like structure 104 protrudes vertically out of the end-effector plane (FIG. 1B).

The temperature sensor was utilized for the following reasons. The FBGs 102 used were small (˜1 mm long) gratings inscribed in the optical fiber 106. A commercial interrogator shined light down the fiber 106, some of which is reflected back from the grating and recorded. The properties (center wavelength shift) of the reflected light depend on the strain experienced by the optical fiber 106. This strain is caused by tissue contact force (grasping) and temperature changes (e.g. air vs tissue). To derive the tissue contact force, it was necessary to know the temperature. In the prototype device, there were four FBGs 102 on the same optical fiber 106. One FBG was made strain-insensitive by bonding it to the metal frame (thus becoming a temperature sensor only). One can separate out the strain-induced changes in wavelength from the other three sensors (strain sensors) by comparing the shift in wavelength experienced by the temperature sensor. A relationship was then established between the force applied to the forceps and the changes in the reflective wavelength due to strain in the other three FBGs. In the prototype, tissue displacement data was also acquired by using the change in angle (collected using angle-data sensors i.e. encoders) when the forceps are grasping the tissue. Together with the force data, one can derive a model describing the reaction of tissue (i.e. strain, or elongation compared to original length, experienced by the tissue) in response to force exerted (detected using the FBGs). In the preliminary study, it was demonstrated that different silicone molds (that are close in hardness compared to tissue) have distinct models and thus can be differentiated using the device 101.

### Force Sensing Principle and Force Calibration

FBG arrays reflect specific wavelengths (Bragg wavelength, λ_{B}) at predefined locations within the fiber allowing for multiple sensing locations on a single fiber. When axial strain is applied, the Bragg grating period is altered, resulting in a change in the wavelength of the reflected light that can be linearly correlated to the applied strain. The shift in the Bragg wavelength caused by strain is (Campanella et al., Sensors (Basel)., vol. 18, no. 9, September 2018):

$$\frac{Δ {λ}_{B}}{{λ}_{B}} = ( 1 - {P}_{e} ) * ϵ \qquad Equation 1$$

where Pₑ is the elasto-optic coefficient and ε is the applied strain. Assuming a constant Young's Modulus for the optical fiber due to small strain, the shift in the Bragg wavelength can be derived as:

$$Δ {λ}_{B} = \frac{{λ}_{B} ( 1 - {P}_{e} )}{A * E} * F = kF \qquad Equation 2$$

where F is the applied force, A is the surface area and E is the Young's Modulus of the fiber. Equation 2 shows that the shift in Bragg wavelength is linear to the applied force with a coefficient k if the contact surface area is held constant.

To derive the linear coefficient k, calibration was performed using a Mark-10 Series 3 Force Gauge (Mark-10 Corporation, NY, USA) with an accuracy of 0.05N mounted on a linear stage. As shown in FIGS. 4A-4C, the force gauge was used to apply normal force to the surface of the puck (FIGS. 4A-4B). In addition, instrument motion was fixed by clamping the pulley system as shown in FIG. 4C. An in-house developed software package (Python) was used to record both data from the force gauge and FBG reflected wavelength detected by the interrogator at a frequency of 10 Hz. Force Calibration was repeated 5 times.

### Benchtop Testing Setup

A benchtop test was performed to ascertain the FBG-integrated instrument's ability to discern different materials based on their mechanical properties. Four silicone blocks of varying hardness were made using off-the-shelf products (Smooth-on (PA, USA) Gel 2, EcoFlex 00-10, EcoFlex 00-30, Mold Start 20T) by mixing 2-part mixture using equal weight (Table 1). These blocks were designed to mimic the mechanical properties of human soft tissue, e.g. EcoFlex 00-30 has been reported to have a Young's Modulus in between rectum and adipose over 0-35% strain (Stokes et al., Proc. Inst. Mech. Eng. Part H J. Eng. Med., vol. 233, no. 1, pp. 114-126, January 2018).

TABLE 1
Silicone Elastomers and Shore Hardness

| Silicone | Shore Hardness |
|---|---|
| Gel 2 | 000-34 |
| EcoFlex 00-10 | 00-10 |
| EcoFlex 00-30 | 00-30 |
| Mold Star 20T | 20A |

Four blocks (size: 38 mm*25 mm*9 mm) were made for each type of silicone elastomer. The silicone rubber mixtures were degassed and cured according to the manufacturer's instructions. The blocks were of the same size and shape and were later spray coated (Plasti Dip, Plasti Dip International, MN, USA) to achieve the same appearance and texture/finish.

The benchtop testing setup is shown in FIG. 5A. The servo motor of FIG. 5B (Lyxnmotion Smart Servo HT-1, Robotshop, Canada) was mounted onto the disc that controls the opening and closing of one side of the grasper. All other instrument motion is prevented by the 3D printed locking mechanism. The silicone blocks were placed on the loading puck 104 and held in place using a 3D-printed holder. The motor was programed to maneuver the non-sensorized side of the grasper from a 90-degree angle to a 21-degree angle over 5 seconds, imitating a grasping motion. All 4 sets of blocks were used, and three measurements were performed on each set. Custom software was used to monitor both the servo motor angle position and FBG central wavelength at 30 Hz.

Data from the loading process were analyzed. Initial contact angle (α₀) and initial wavelength (λ_{B}) were defined as the angle and Bragg wavelength at 10% of the maximum wavelength shift during loading. Δα and Δλ_{B} were then calculated accordingly. Strain is calculated as:

$$Strain = \frac{Δ α}{{α}_{0}} \qquad Equation 3$$

Force can be calculated using Equation (4) obtained from calibration results.

### Human Subject Experiment

To test the effect of the loss of haptic feedback when performing robotic surgery, a total of 5 novices and one expert Da Vinci user were tasked with sorting the set of four blocks by hardness. Initially, each subject undertook this task four times using a Da Vinci Si with a standard Da Vinci Prograsp graspers. The test was then repeated via manual palpation of the blocks. All research involving human subjects was approved by the Institutional Review Board.

### Results

### Sensor Manufacturing

The sensor was successfully manufactured and integrated into the Da Vinci surgical instrument. The FBG-integrated sensor was robust and tolerated both force calibration and benchtop testing with good repeatability. FBG_1, FBG_2 and FBG_3 behaved similarly in both force calibration and benchtop testing and therefore only FBG_2 data is shown for both force calibration and benchtop testing results below.

### Force Calibration Results

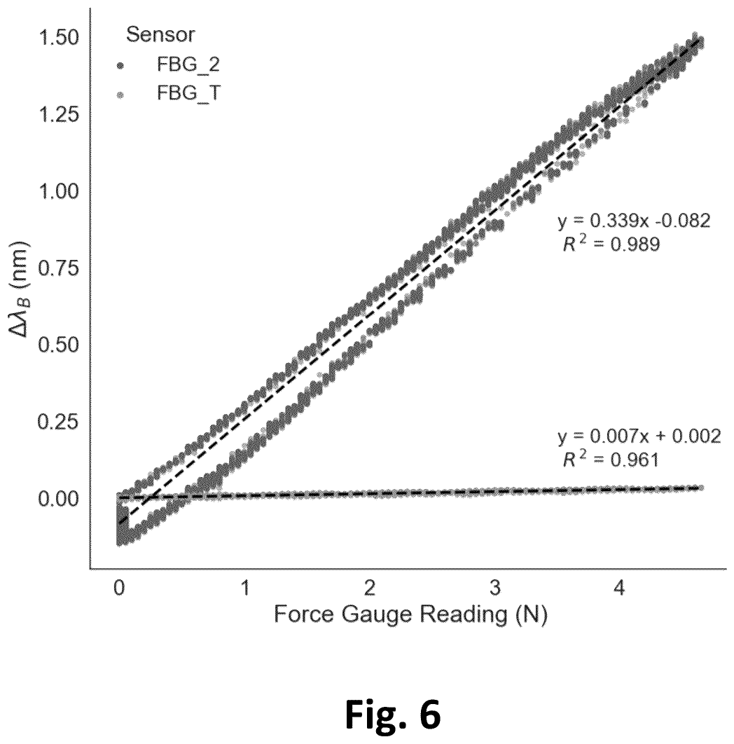

The shifts in Bragg wavelengths in each FBG sensor 102 were calculated by subtracting the central wavelength by the baseline (average central wavelength when no force was applied). Force calibration was carried out by mapping Force Gauge reading with the corresponding shift in Bragg wavelength (FIG. 6). Data across all five trials are consistent, showing good repeatability and a strong linear correlation (r²>0.961 for all sensors), The wavelength shifts of FBG_2 displayed a correlation with the applied force as predicted by Equation (2). Coefficient k was determined to be 0.339 for FBG_2, such that:

$$F = \frac{Δ {λ}_{B}}{0.339} \qquad Equation 4$$

In contrast, FBG_T is effectively strain-shielded as designed, thus, facilitating future use as a temperature compensation sensor.

However, while the force calibration curve of the sensor is mostly linear, it shows distinct loading and unloading curves indicating hysteresis, likely due to the viscoelasticity of PDMS. Future iterations of this sensor will deploy a different substrate and a new puck design to accommodate a wider force sensing range with higher precision.

### Benchtop Testing Results

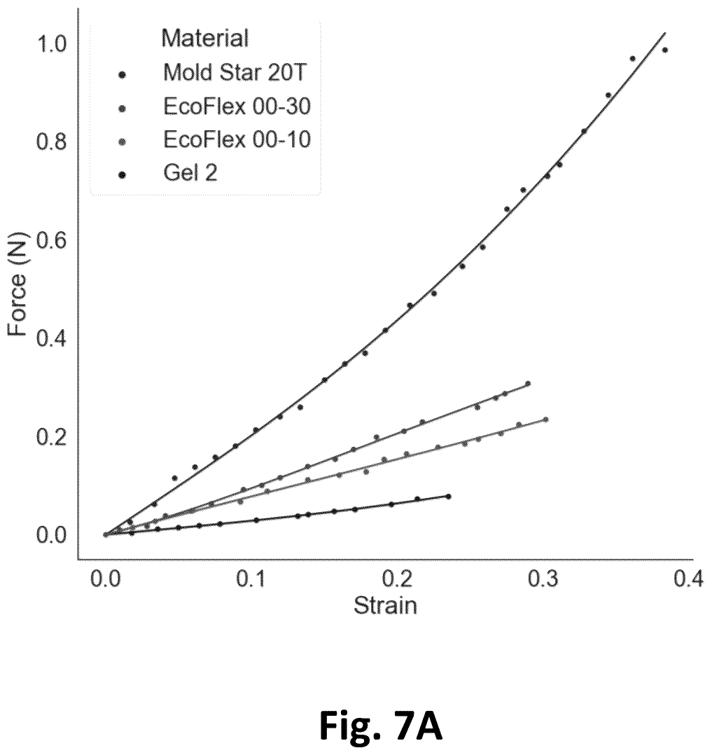

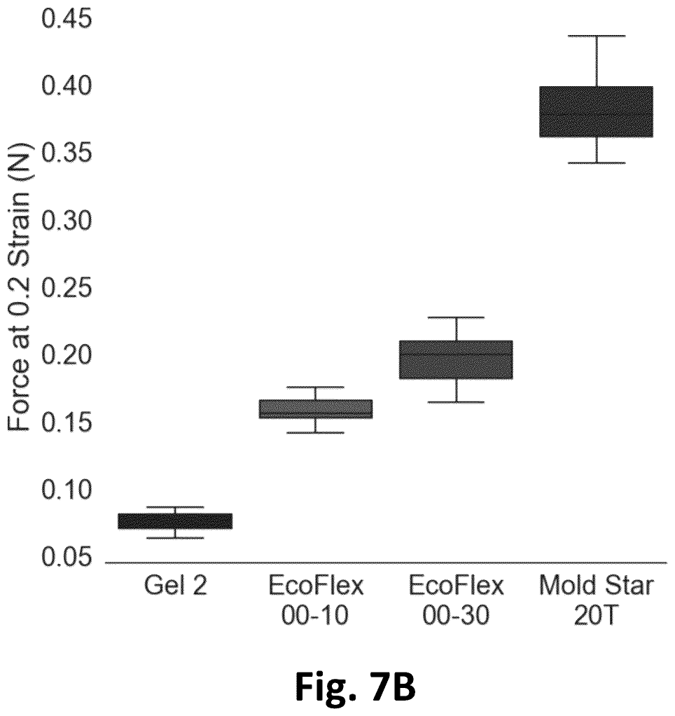

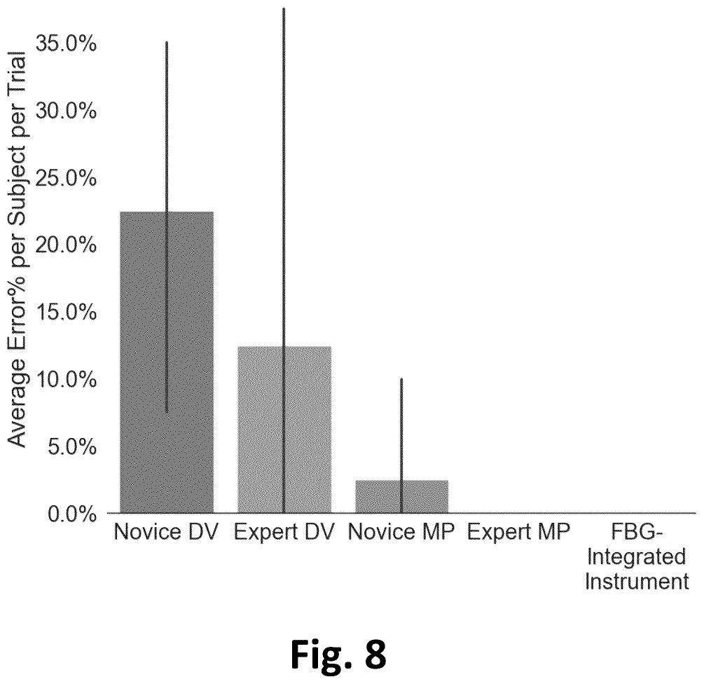

The FBG-integrated device 101 and system 100 was successful in identifying all four silicone blocks without any errors (FIG. 8). For each trial, a force-strain curve was plotted for each material and second-order Ogden Model for uniaxial loading (Ogden et al., Non-linear elastic deformations. Courier Corporation, 1997; Marechal et al., Soft Robot., vol. 8, no. 3, pp. 284-297, June 2021) was used for the trend line. An exemplary plot can be seen in FIG. 7A. The force-strain curve produced during material loading (grasper closing) shows a distinct curve for each material and a much higher slope for stiffer materials as expected. In this test, materials were easily identified by quantifying the force needed to produce a strain of 0.2 (FIG. 7B). Force at 0.20 Strain (mean±standard deviation) is calculated for all four materials: 0.076±0.008N for Gel 2, 0.159±0.009N for EcoFlex 00-10, 0.197±0.020N for EcoFlex 00-30 and 0.383±0.030N for Mold Star 20T. The low standard deviation of the measurements indicated the high precision of the sensor.

### Human Subject Experiment Results

When sorting molds with the Da Vinci surgical robot, the average error per subject per trial was 22.5% for novices and 12.5% for the expert Da Vinci user (FIG. 8). In contrast, the same users exhibited substantially improved performance when directly sorting molds using manual palpation with errors of 2.5% and 0% recorded for the novices and expert respectively. During the experiment, subjects expressed that they were confident about determining the hardest material, but had great difficulty discerning the 3 softer materials, which corresponds to the results shown in FIG. 7B. This experiment confirmed that without haptic feedback, a loss of tissue mechanical property information occurs when surgical robots were used. While the performance is significantly better when the sorting was done using manual palpation, the sense of touch by its nature cannot be quantified and is subjective, therefore it is not “foolproof”. As shown in FIG. 8, sorting molds using manual palpation still resulted in errors in novices; in contrast, the FBG-integrated instrument was able to provide repeatable quantitative data to identify the four silicone blocks and made no errors in all trials. This experiment clearly showcased the system's 100 ability to surpass the limit of human mechanoreceptors.

With the success of the first experiments, additional experiments were performed. Six silicone blocks of varying hardness (Table 2) were made using off-the-shelf products (Smooth-on (PA, USA) Gel 2, EcoFlex 00-10, EcoFlex 00-30, Dragon Skin FX Pro, Dragon Skin 10 Medium, Mold Start 20T). Three blocks were made for each type of elastomer following the aforementioned method described above. In total, three sets of six blocks were used in the following experiment with one set being the training set and the remaining two as test sets.

TABLE 2
Silicone Elastomers and Shore Hardness

| Silicone | Shore Hardness |
|---|---|
| Gel 2 | 000-34 |
| EcoFlex 00-10 | 00-10 |
| EcoFlex 00-30 | 00-30 |
| Dragon Skin FX Pro | 2A |
| Dragon Skin 10 | 10A |
| Medium | |
| Mold Star 20T | 20A |

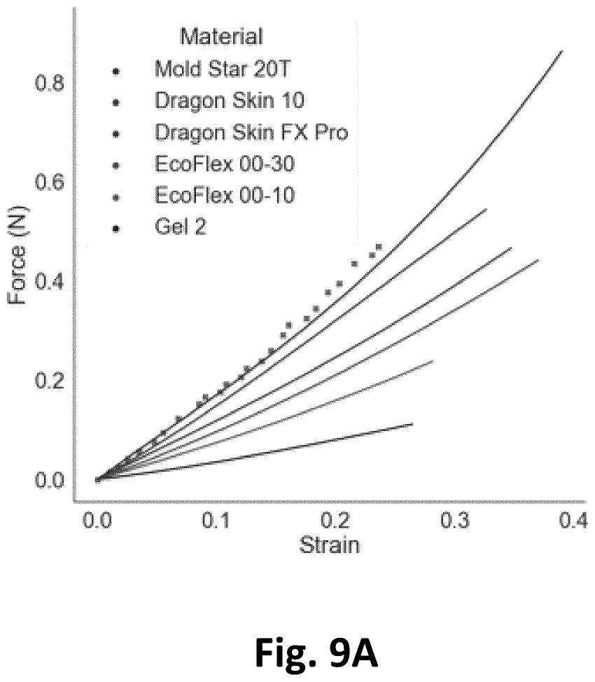

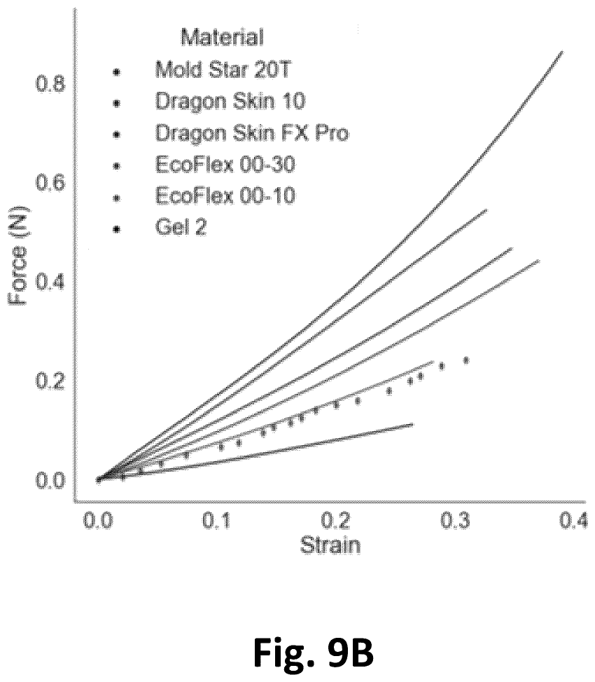

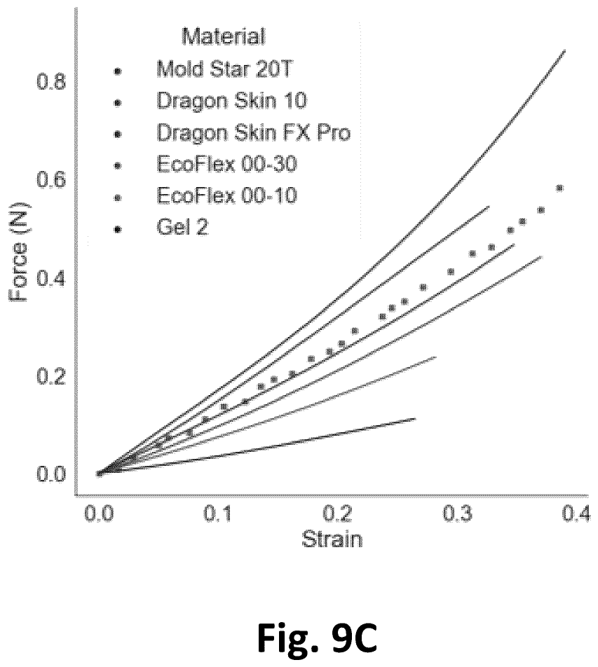

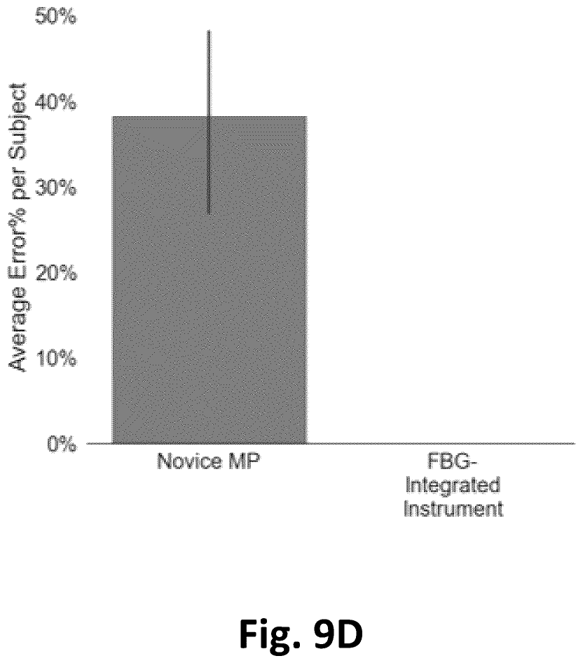

To establish a library of tissue models, force-strain models for each silicone type were produced using the training set. Briefly, three measurements were taken for each silicone type using the FBG-integrated sensor, similar to the method discussed above. A force-strain curve was plotted for each silicone type and fitted to a second-order Ogden model for uniaxial loading. To identify tissue, it is necessary to compare real-time measurements to the library of tissue models. This approach was successfully demonstrated by acquiring measurements of randomly chosen blocks from the two test sets (12 silicone blocks total). For each measurement, the data set was fitted to each of the six tissue library models established above and a least-squares comparison was used to find the best-fitted model. FIGS. 9A-9C show measurements from three different samples and how each data set matches closely to its corresponding model in the tissue library. The experiment was repeated 20 times and the sensor was able to successfully categorize the hardness level of each block.

To test if manual palpation can perform as well as the sensor, a total of eight subjects were recruited. The subjects attempted to create their own mental tissue library by pinching each of the six training set blocks between index finger and thumb. These blocks were labelled in order of hardness and the subjects were allowed to continually train for 90 seconds. Subjects were then presented with one randomly chosen silicone block from the two test sets. They were allowed to palpate the test block once and determine its hardness level. Note, they were not allowed to palpate the test set again for comparison and instead had to attempt to identify the tissue block based on the metal tissue library established from the training set. The experiment was repeated 20 times for each subject. The average error rate was 38.4% across all subjects which is substantially inferior to the FBG-integrated instrument which exhibited no error (FIG. 9D).

These experiments, together with the tissue sorting experiments, demonstrate that while tissue mechanical properties can be differentiated through manual palpation, the information is hard to retain. The sensor has shown its potential in establishing a much-needed tissue mechanical database based on quantitative and subjective data that would be much more informative and beneficial compared to tactile feedback in a surgical environment.

### CONCLUSION

In these studies, a novel sensor utilizing FBG was integrated directly into existing surgical instruments to detect tissue mechanical properties during RMIS procedures. The low-cost, low-profile and sterilizable sensor is a candidate for future clinical translation. This proof-of-concept study demonstrated the validity of this design. The Bragg wavelength shift of the sensor is linear to the force applied at the puck with a high coefficient of determination for all sensors. Additionally, the sensor was able to successfully differentiate silicone blocks with mechanical properties representative of human tissues. Moreover, the performance was superior to that achieved by human subjects using a conventional surgical robot and comparable to direct palpation by hand. In a surgical setting where hundreds if not thousands of palpation or grasping motions happen, it would be unreasonable to assume that surgeons would be able to recall minute differences in tissue hardness without any quantitative data to rely on and this novel sensor can fill that gap to allow more informed decision-making during robotic surgery.

### Computing Environment

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C#, Objective-C, Java, Javascript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

FIG. 10 and the following discussion are intended to provide a brief, general description of a suitable computing environment in which the invention may be implemented. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

FIG. 10 depicts an illustrative computer architecture for a computer 1000 for practicing the various embodiments of the invention. The computer architecture shown in FIG. 10 illustrates a conventional personal computer, including a central processing unit 1050 (“CPU”), a system memory 1005, including a random-access memory 1010 (“RAM”) and a read-only memory (“ROM”) 1015, and a system bus 1035 that couples the system memory 1005 to the CPU 1050. A basic input/output system containing the basic routines that help to transfer information between elements within the computer, such as during startup, is stored in the ROM 1015. The computer 1000 further includes a storage device 1020 for storing an operating system 1025, application/program 1030, and data.

The storage device 1020 is connected to the CPU 1050 through a storage controller (not shown) connected to the bus 1035. The storage device 1020 and its associated computer-readable media, provide non-volatile storage for the computer 1000. Although the description of computer-readable media contained herein refers to a storage device, such as a hard disk or CD-ROM drive, it should be appreciated by those skilled in the art that computer-readable media can be any available media that can be accessed by the computer 1000.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the invention, the computer 1000 may operate in a networked environment using logical connections to remote computers through a network 1040, such as TCP/IP network such as the Internet or an intranet. The computer 1000 may connect to the network 1040 through a network interface unit 1045 connected to the bus 1035. It should be appreciated that the network interface unit 1045 may also be utilized to connect to other types of networks and remote computer systems.

The computer 1000 may also include an input/output controller 1055 for receiving and processing input from a number of input/output devices 1060, including a keyboard, a mouse, a touchscreen, a camera, a microphone, a controller, a joystick, or other type of input device. Similarly, the input/output controller 1055 may provide output to a display screen, a printer, a speaker, or other type of output device. The computer 1000 can connect to the input/output device 1060 via a wired connection including, but not limited to, fiber optic, ethernet, or copper wire or wireless means including, but not limited to, Bluetooth, Near-Field Communication (NFC), infrared, or other suitable wired or wireless connections.

As mentioned briefly above, a number of program modules and data files may be stored in the storage device 1020 and RAM 1010 of the computer 1000, including an operating system 1025 suitable for controlling the operation of a networked computer. The storage device 1020 and RAM 1010 may also store one or more applications/programs 1030. In particular, the storage device 1020 and RAM 1010 may store an application/program 1030 for providing a variety of functionalities to a user. For instance, the application/program 1030 may comprise many types of programs such as a word processing application, a spreadsheet application, a desktop publishing application, a database application, a gaming application, internet browsing application, electronic mail application, messaging application, and the like. According to an embodiment of the present invention, the application/program 1030 comprises a multiple functionality software application for providing word processing functionality, slide presentation functionality, spreadsheet functionality, database functionality and the like.

The computer 1000 in some embodiments can include a variety of sensors 1065 for monitoring the environment surrounding and the environment internal to the computer 1000. These sensors 1065 can include a Global Positioning System (GPS) sensor, a photosensitive sensor, a gyroscope, a magnetometer, thermometer, a proximity sensor, an accelerometer, a microphone, biometric sensor, barometer, humidity sensor, radiation sensor, or any other suitable sensor.

The following publications are each hereby incorporated herein by reference in their entirety:
G. F. Buess, M. O. Schurr, and S. C. Fischer, “Robotics and allied technologies in endoscopic surgery,” Arch. Surg., vol. 135, no. 2, pp. 229-235, 2000.
J. D. Brown, J. Rosen, L. Chang, M. N. Sinanan, and B. Hannaford, “Quantifying Surgeon Grasping Mechanics in Laparoscopy Using the Blue DRAGON System,” Stud. Health Technol. Inform., vol. 98, pp. 34-36, 2004.
W. Y. Lau, C. K. Leow, and A. K. C. Li, “History of endoscopic and laparoscopic surgery,” World J. Surg., vol. 21, no. 4, pp. 444-453, 1997.
M. O. Schurr, G. Buess, B. Neisius, and U. Voges, “Robotics and telemanipulation technologies for endoscopic surgery. A review of the ARTEMIS project. Advanced Robotic Telemanipulator for Minimally Invasive Surgery,” Surg. Endosc., vol. 14, no. 4, pp. 375-381, April 2000.
K. Moorthy et al., “Dexterity enhancement with robotic surgery”, Surg. Endosc., vol. 18, no. 5, pp. 790-795, 2004.
Y. Munz et al., “The benefits of stereoscopic vision in roboticassisted performance on bench models,” Surg. Endosc., vol. 18, no. 4, pp. 611-616, April 2004.
K. H. Sheetz, J. Claflin, and J. B. Dimick, “Trends in the Adoption of Robotic Surgery for Common Surgical Procedures,” JAMA Netw. Open, vol. 3, no. 1, pp. e1918911-e1918911, January 2020.
M. Li, H. Liu, J. Li, L. D. Seneviratne, and K. Althoefer, “Tissue stiffness simulation and abnormality localization using pseudohaptic feedback,” Proc.-IEEE Int. Conf. Robot. Autom., pp. 5359-5364, 2012.
E. J. Hanly and M. A. Talamini, “Robotic abdominal surgery,” Am. J. Surg., vol. 188, no. 4A Suppl, pp. 19-26, 2004.
S. Mckinley et al., “A single-use haptic palpation probe for locating subcutaneous blood vessels in robot-assisted minimally invasive surgery,” IEEE Int. Conf. Autom. Sci. Eng., vol. 2015-October, pp. 1151-1158 October 2015.
M. T. Perri, A. L. Trejos, M. D. Naish, R. V. Patel, and R. A. Malthaner, “New tactile sensing system for minimally invasive surgical tumour localization,” Int. J. Med. Robot., vol. 6, no. 2, pp. 211-220, June 2010.
H. Xie, H. Liu, S. Luo, L. D. Seneviratne, and K. Althoefer, “Fiber optics tactile array probe for tissue palpation during minimally invasive surgery,” IEEE Int. Conf. Intell. Robot. Syst., pp. 2539-2544, 2013.
C. Pacchierotti, D. Prattichizzo, and K. J. Kuchenbecker, “Cutaneous feedback of fingertip deformation and vibration for palpation in robotic surgery,” IEEE Trans. Biomed. Eng., vol. 63, no. 2, pp. 278-287, February 2016.
C. E. Campanella, A. Cuccovillo, C. Campanella, A. Yurt, and V. M. N. Passaro, “Fibre Bragg Grating Based Strain Sensors: Review of Technology and Applications,” Sensors (Basel)., vol. 18, no. 9, September 2018.
W. E. Stokes, D. G. Jayne, A. Alazmani, and P. R. Culmer, “Development of a Physical Simulation of the Human Defecatory System for the Investigation of Continence Mechanisms,” Proc. Inst. Mech. Eng. Part H J. Eng. Med., vol. 233, no. 1, pp. 114-126, January 2018.
R. Ogden, Non-linear elastic deformations. Courier Corporation, 1997.
L. Marechal, P. Balland, L. Lindenroth, F. Petrou, C. Kontovounisios, and F. Bello, “Toward a Common Framework and Database of Materials for Soft Robotics,” Soft Robot., vol. 8, no. 3, pp. 284-297, June 2021.
Abiri A, Juo Y Y, Tao A, Askari S J, Pensa J, Bisley J W, Dutson E P, Grundfest W S. Artificial palpation in robotic surgery using haptic feedback. Surg Endosc. 2019 April; 33 (4): 1252-1259. doi: 10.1007/s00464-018-6405-8. Epub 2018 Sep. 5. PMID: 30187198; PMCID: PMC6401328.
Abiri A, Tao A, LaRocca M, et al. Visual-perceptual mismatch in robotic surgery. Surg Endosc. 2017; 31 (8): 3271-3278. doi: 10.1007/s00464-016-5358-z
Abiri A, Pensa J, Tao A, et al. Multi-Modal Haptic Feedback for Grip Force Reduction in Robotic Surgery. 2019:1-10. doi: 10.1038/s41598-019-40821-1
Wottawa C R. An Investigation into the Benefits of Tactile Feedback for Laparoscopic, Robotic, and Remote Surgery. 2013
Wottawa C R, Genovese B, Nowroozi B N, et al. Evaluating tactile feedback in robotic surgery for potential clinical application using an animal model. Surg Endosc. 2016; 30 (8): 3198-3209. doi: 10.1007/s00464-015-4602-2
PCT Patent App. PCT/US2019/034630 filed May 30, 2019, titled “Haptic feedback sensor and method of making the same.”
PCT Patent App. PCT/US2018/055569 filed Oct. 12, 2018, titled “Multi-modal haptic feedback system.”

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A device for tissue mechanical property detection during robotic surgery comprising:
a sensor frame having proximal and distal ends and a length therebetween;
a force sensor disposed along the length of the sensor frame; and
a displacement sensor configured to measure a position of the sensor frame.

2. The device of claim 1, further comprising a temperature sensor.

3. The device of claim 1, further comprising a loading puck near the distal end of the sensor frame.

4. The device of claim 1, wherein the sensor frame is configured as surgical forceps.

5. The device of claim 1, wherein the force sensor comprises one or more fiber Bragg grating (FBG) sensors.

6. The device of claim 1, wherein the force sensor comprises one or more piezoelectric sensors.

7. The device of claim 1, wherein the force sensor comprises one or more capacitive sensors.

8. The device of claim 1, wherein the force sensor comprises a multiplexed sensor.

9. The device of claim 1, wherein the displacement sensor comprises an angle encoder, a camera, or a stereoscope.

10. A system for tissue mechanical property detection during robotic surgery comprising:
a sensor frame having proximal and distal ends and a length therebetween;
a force sensor disposed along the length of the sensor frame;
a displacement sensor configured to measure a position of the sensor frame; and
a robotic grasping arm, wherein the sensor frame is positioned as an end-effector of the robotic grasping arm.

11. The system of claim 10, further comprising a computing system communicatively connected to the force and displacement sensors, comprising a processor and a non-transitory computer-readable medium with instructions stored thereon, which when executed by the processor, perform steps comprising:
obtaining force data via the force sensor;
obtaining tissue displacement data via the displacement sensor;
applying the obtained force and displacement data to a tissue specific model representing the strain experienced by tissue in response to an external force; and
identifying at least one mechanical property of the tissue based on the model output.

12. A method of identifying tissue mechanical properties during robotic surgery, comprising the steps of:
providing the tissue mechanical property detection system of claim 10;
grasping a tissue with the end-effector of the robotic grasping arm such that the sensor device engages the tissue;
obtaining force data via the force sensor;
obtaining tissue displacement data via the displacement sensor;
applying the obtained force and displacement data to a tissue specific model representing the strain experienced by the tissue in response to an external force; and
identifying at least one mechanical property of the tissue based on the model output.

13. The method of claim 12, further comprising obtaining temperature data from a temperature sensor.

14. The method of claim 12, further comprising identifying the type of tissue grasped based on the identification of the at least one tissue mechanical property.

15. The method of claim 14, wherein the at least one tissue mechanical property is determined based on a force-strain model.

16. The method of claim 14, wherein the identification of the type of tissue is based on comparing the identified tissue mechanical property against a library including tissue types and their mechanical properties, where the library is built via training data sets comprising a plurality of tissue types with known tissue properties.

16. The method of claim 12, further comprising providing feedback to an end-user or autonomous system.

17. The method of claim 16, wherein the feedback comprises a vibration, a visual cue, or an auditory cue.

18. The method of claim 17, wherein the feedback is provided in less than 1 second, less than 0.5 seconds, or in less than 0.1 seconds after grasping the tissue.

19. The method of claim 12, wherein tissue displacement is inferred by recording the position of the graspers with a displacement sensor.

20. The method of claim 19, wherein the displacement sensor comprises an angle encoder, a camera, or a stereoscope.
